(19) United States
(12) Patent Application Publication
(10) Pub. No.: US 2016/0081414 A1
(43) Pub. Date: Mar. 24, 2016

(54) CPSIA-COMPLIANT, SOUND-DEADENING INFANT SLEEP HAT

(71) Applicant: Hush Baby, LLC, Meza, AZ (US)

(72) Inventors: Jaycee Bybee, Meza, AZ (US); Erin Urrutia, Meza, AZ (US)

(21) Appl. No.: 14/863,335

(22) Filed: Sep. 23, 2015

Related U.S. Application Data
(60) Provisional application No. 62/054,336, filed on Sep. 23, 2014.

(51) Int. Cl. A42B 1/06; A61F 11/14

(52) U.S. Cl. CPC A42B 1/068; A61F 11/14

(57) ABSTRACT

A sound-deadening infant sleep hat that is compliant with the Consumer Product Safety Improvement Act of 2008 (“CPSIA”) is provided. In various embodiments, the CPSIA-compliant, sound-deadening infant sleep hat may include a non-rigid outer shell having an inner surface upon which two non-rigid ear pieces are disposed opposite one another. The non-rigid ear pieces may each be at least partially composed of one or more sound-deadening materials in a combined or layered arrangement. The one or more sound-deadening materials may include a viscoelastic sound-deadening foam and a sound-deadening material composed at least partially of one or more fibers.

### CROSS-REFERENCE TO RELATED APPLICATION

The present application claims the priority benefit of U.S. provisional application No. 62/054,336 filed Sep. 23, 2014 and entitled “CPSIA-Compliant, Sound-Deadening Infant Sleep Hat,” the disclosure of which is incorporated herein by reference.

### BACKGROUND

1. Field of the Disclosure

The present disclosure relates to infant sleepwear. More particularly, the present disclosure concerns a sound-deadening infant sleep hat.

2. Description of the Related Art

Every caretaker knows that an infant's ability to sleep soundly is important for numerous reasons. Infants require adequate sleep to grow and remain healthy during one of the most critical developmental phase of life. Caretakers (e.g., parents, grandparents, nursery workers, babysitters, etc.) likewise need infants to sleep soundly so that the caretakers themselves can get a good night's rest. An infant's ability to sleep soundly also increases the probability that caretakers can take the infant to public venues without the fear of inconveniencing others or having to leave altogether due to sleep-related bouts of crying.

Despite its importance, sound sleep does not come easily for some infants. The modern world is full of noises that interrupt important sleep cycles or prevent infants from falling asleep altogether. Worse yet, some noises can irreparably damage an infant's sensitive hearing. According to the American Academy of Pediatrics and the Children's Hearing Institute, noises over 85 decibels (dB) are dangerous for adult ears, let alone infant ears containing comparatively smaller ear canals. Studies suggest that the smaller scale of infant ear canals causes infant ears to automatically amplify high-frequency sounds. Premature infants or infants born with colic or certain genetic syndromes are also particularly prone to noise sensitivity and over stimulation. As a result, in some cases an infant may actually hear a given noise at a higher decibel level than an adult simultaneously hearing the same noise.

In light of those considerations unique to infant ears, the Sound Study Group of the National Resource Center recommends limiting noise exposure to 50 dB averaged over a one-hour period for infants in hospital nurseries or similar environments. Many every-day noises, however, far exceed 50 dB. Some common examples include noises from: a vacuum cleaner (70 dB), an ambulance siren (120 dB), firecrackers (120 to 140 dB), motorcycles (120 to 140 dB), certain toys (90 dB), restaurants (80 to 95 dB), microwave alarms and alarm clocks (80 dB), city traffic (80 dB), and television (90 to 120 dB). Because infants are routinely exposed to noises in exceed of 50 dB, sleeping soundly and safely is a real challenge in some instances.

Previous attempts to facilitate infant sleep are inadequate. Studies indicate, for example, that white noise sleep machines can damage infant ears when used over extended periods of time. Existing sound-deadening technologies are likewise unsuitable for sleeping infants. For example, although protective headphones have existed for some time (e.g., those offered by David Clark Company of Worcester, Mass.), they rely on forming a seal around each ear. To form that seal, the headphones must be tension-biased so that they apply pressure to the head region located proximate to the ear. Over time, that pressure leads to discomfort even for adults. Infants, whose ears and developing heads are much more sensitive than those of adults, cannot sleep soundly through such discomfort. Pressure-induced discomfort aside, protective headphones are not appropriately sized for infants. Moreover, they are rigid and unsuitable as sleepwear because they do not allow an infant's head to comfortably rest at the proper angle needed to facilitate sleep.

Importantly, sleepwear for infants must also pass specific children's sleepwear regulations implemented by the U.S. Consumer Product Safety Commission in accordance with the Consumer Product Safety Improvement Act of 2008 (CPSIA). See 16 C.F.R. Parts 1615 and 1616. In the context of this disclosure, the phrase “CPSIA-compliant” refers to a product that is compliant with children's sleepwear regulations implemented in accordance with the CPSIA.

Previously attempted solutions at reducing noise for infants do not account for the 2008 passage of the CPSIA, ignore important safety considerations, and are likely unsuitable as infant sleepwear under the CPSIA. Products made of a warm fleece material, for example, can cause overheating when used indoors. Studies have shown that infants have a higher chance of experiencing sudden infant death syndrome (commonly known as “SIDS”) when exposed to excessively high temperatures during sleep. Garments that contain tassels, straps, or clips can present a choking hazard when used as sleepwear.

### SUMMARY OF THE CLAIMED INVENTION

Embodiments of a sound-deadening infant sleep hat are claimed.

In a first embodiment, a sound-deadening infant sleep hat includes a non-rigid outer shell having an inner surface. The sound-deadening infant sleep hat includes two non-rigid ear pieces disposed opposite one another on the inner surface of the non-rigid outer shell. The non-rigid ear pieces are each composed at least partially of at least one sound-deadening material.

In a second claimed embodiment, a sound-deadening infant sleep hat includes a non-rigid outer shell having an inner surface. The sound-deadening infant sleep hat includes two non-rigid ear pieces disposed opposite one another on the inner surface of the non-rigid outer shell. The non-rigid ear pieces are each at least partially composed of at least a first sound-deadening material and a different second sound-deadening material.

In a third claimed embodiment, a sound-deadening infant sleep hat includes a non-rigid outer shell having an inner surface. The sound-deadening infant sleep hat includes two non-rigid ear pieces disposed opposite one another on the inner surface of the non-rigid outer shell. The non-rigid ear pieces are each at least partially composed of at least a first layer of viscoelastic sound-deadening foam and a second layer of sound-deadening material composed at least partially of one or more fibers. The sound-deadening infant sleep hat further includes a mesh band disposed over the two non-rigid ear pieces on the inner surface of the non-rigid outer shell.

### BRIEF DESCRIPTION OF DRAWINGS

FIGS. 1, 2, and 3 are, respectively, perspective, top, and side views of an exemplary CPSIA-compliant, sound-deadening infant sleep hat.

FIG. 4 is side view of an exemplary CPSIA-compliant, sound-deadening infant sleep hat after having been flipped inside-out to reveal an inner surface of a non-rigid outer shell.

FIG. 5A is a perspective view of an exemplary CPSIA-compliant, sound-deadening infant sleep hat showing an inner surface of a panel of a non-rigid outer shell.

FIG. 5B is a perspective view of an exemplary CPSIA-compliant, sound-deadening infant sleep hat showing the same components as FIG. 5A with the non-rigid inner band fully displayed.

FIGS. 6A through 6D are, respectively, front, side, perspective, and bottom views of an exemplary panel of a non-rigid outer shell.

FIGS. 7A through 7E are, respectively, side, zoomed side, front, perspective, and top views of an exemplary panel of a non-rigid outer shell combined with an exemplary non-rigid inner band.

FIGS. 8A through 8D are, respectively, front, side, top, and perspective views of an exemplary non-rigid ear piece.

FIGS. 9, 10, 11A, 11B, and 11C are, respectively, perspective, front, top, zoomed top, and side views of a panel of a non-rigid outer shell combined with a non-rigid inner band and a non-rigid ear piece.

FIG. 12 is a graph that illustrates the sound-deadening characteristics of an exemplary CPSIA-compliant, sound-deadening infant sleep hat.

FIG. 13 is another graph that illustrates the sound-deadening characteristics of an exemplary CPSIA-compliant, sound-deadening infant sleep hat.

### DETAILED DESCRIPTION

A sound-deadening infant sleep hat that is compliant with the Consumer Product Safety Improvement Act of 2008 (“CPSIA”) is provided. In various embodiments, the CPSIA-compliant, sound-deadening infant sleep hat may include a non-rigid outer shell having an inner surface upon which two non-rigid ear pieces are disposed opposite one another. The non-rigid ear pieces may each be at least partially composed of one or more sound-deadening materials in a combined or layered arrangement. The one or more sound-deadening materials may include a viscoelastic sound-deadening foam and a sound-deadening material composed at least partially of one or more fibers (e.g., a polyester fiber blend, reinforced polyester fibers, and the like). In some embodiments, by combining viscoelastic energy-absorbing foam with one or more materials at least partially composed of one or more fibers (e.g., a polyester fiber blend, reinforced polyester fibers, and the like), the CPSIA-compliant, sound-deadening infant sleep hat disclosed herein may be customized to deaden sound over a wide range of frequencies (e.g., between around 1 dB to around 20 dB over a frequency of at least around 850 Hz to at least around 20 kHz) or to intelligently deaden sound only at desired frequencies.

The various embodiments described in the present disclosure are exemplary in nature and were selected to most clearly convey the technology, but they are in no way exhaustive and should not be construed as such. After reading the following description, persons of ordinary skill in the art will readily recognize that many other embodiments and variations are possible and conveyed within the scope of the present disclosure. For purposes of the present disclosure, any dimension described should be interpreted as including a reasonable range or tolerance on either side of the particular dimension specified for illustrative purposes. Additionally, persons of ordinary skill in the art will readily recognize that the dimensions described herein are merely illustrative and that many other possible dimensions are possible and conveyed within the scope of the present disclosure.

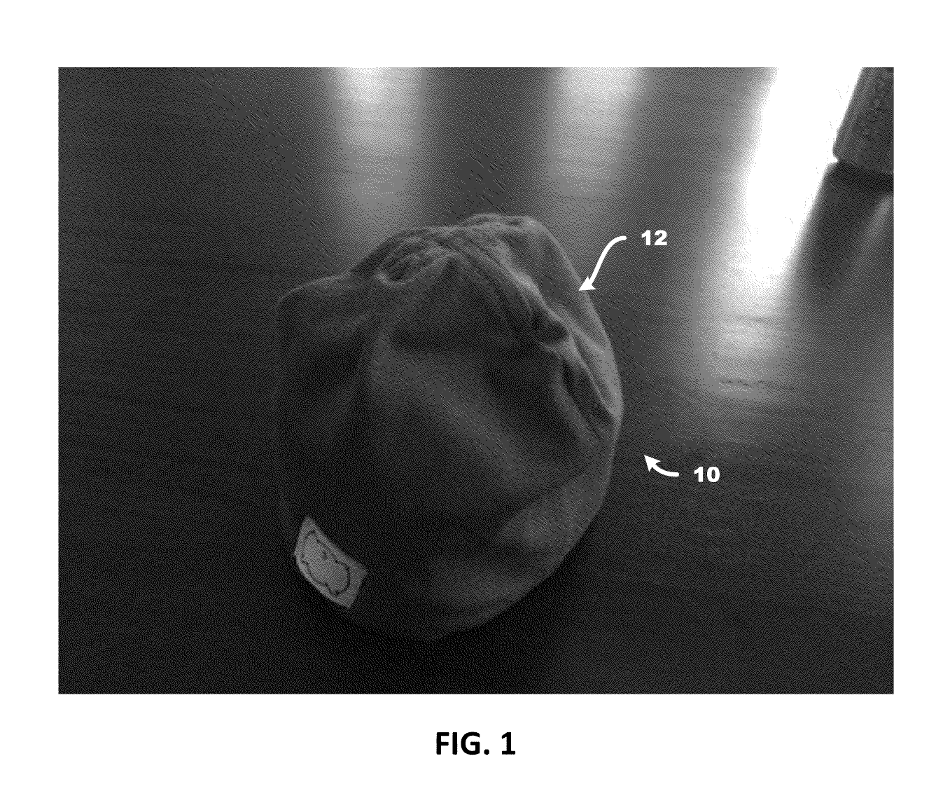

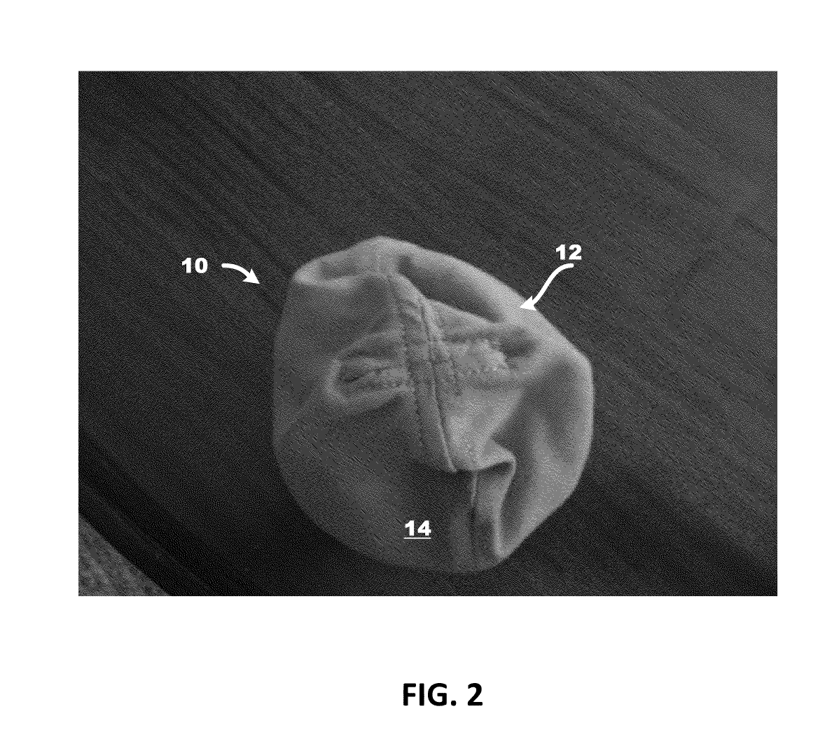

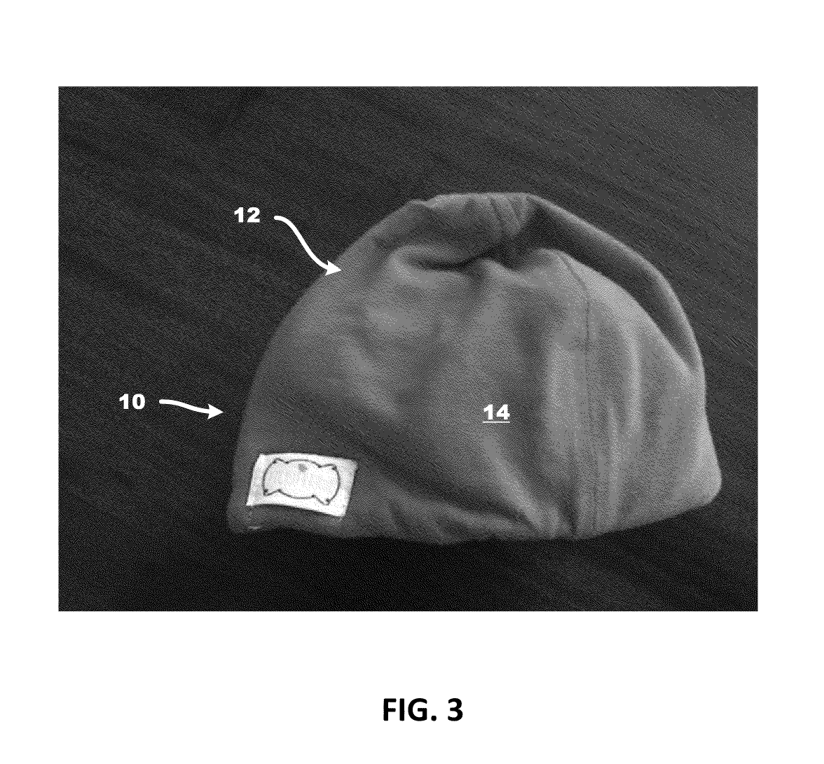

FIGS. 1, 2, and 3 are, respectively, perspective, top, and side views of an exemplary CPSIA-compliant, sound-deadening infant sleep hat. The infant sleep hat contains one or more sound-deadening materials and may be suitably sized and constructed of non-rigid materials to be comfortably and safely worn by a sleeping infant. The infant sleep hat deadens sound and reduces noise that could otherwise wake a sleeping infant or harm the infant's hearing. In various possible embodiments, the sound-deadening materials may include a viscoelastic energy-absorbing foam, a viscoelastic energy-absorbing foam combined with or layered in combination with a one or more fibers (e.g., a polyester fiber blend, reinforced polyester fibers, and the like) material, a viscoelastic energy-absorbing foam combined with or layered in combination with a fabric material, or a viscoelastic energy-absorbing foam combined with or layered in combination with other non-rigid materials that exhibit sound-deadening characteristics.

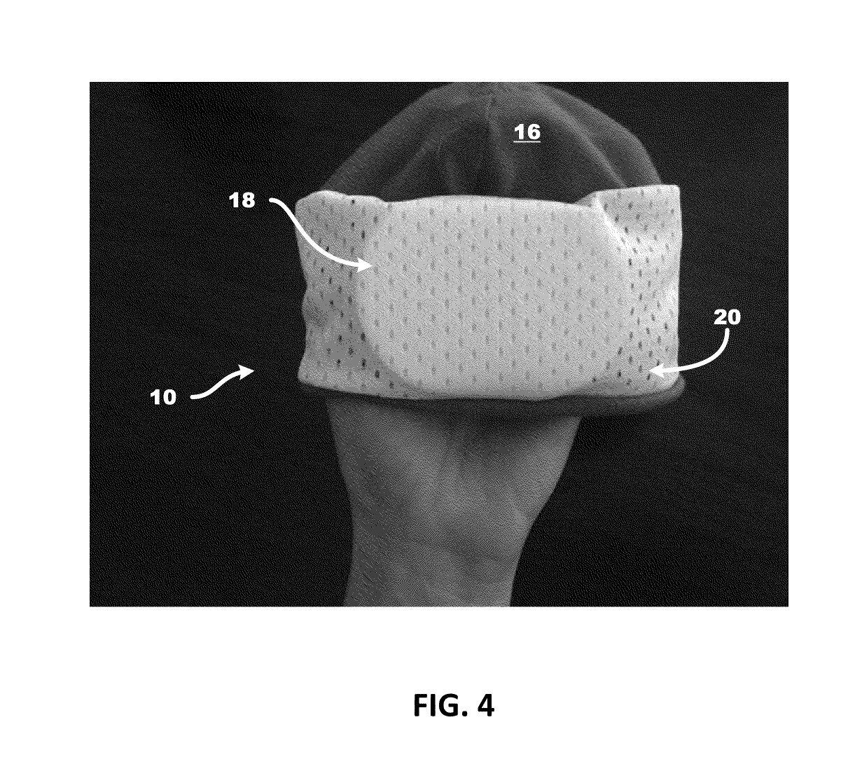

As shown in FIGS. 1, 2, and 3, in one embodiment a CPSIA-compliant, sound-deadening infant sleep hat 10 may include a non-rigid outer shell 12. Non-rigid outer shell 12 may include an outer surface 14 and an inner surface 16 (shown in FIG. 4). Non-rigid outer shell 12 may be manufactured from any non-rigid material suitable for comfortable sleep activities, such as cotton, polyester, or a blend (e.g., a cotton-rayon blend). As shown in FIGS. 1, 2, and 3, CPSIA-compliant, sound-deadening infant sleep hat 10 may be free of any tassels, straps, or headband-like components that would present a choking hazard and compromise compliance with the CPSIA. As discussed below, CPSIA-compliant, sound-deadening infant sleep hat 10 may also include as few components as possible to reduce manufacturing costs and to avoid an unnecessary increase in the likelihood of structural integrity over time.

FIG. 4 is side view of an exemplary CPSIA-compliant, sound-deadening infant sleep hat after having been flipped inside-out to reveal inner surface 16 of non-rigid outer shell 12. As shown in FIG. 4, in one embodiment CPSIA-compliant, sound-deadening infant sleep hat 10 may include two non-rigid ear pieces 18 that each includes one or more sound-deadening materials. As discussed further below, in some embodiments non-rigid ear pieces 18 may each include a solid area of foam or other soft material that deadens sound without requiring a seal around the infant's ear. Non-rigid ear pieces 18 may include a plurality of sound-deadening materials (e.g., in a combined or layered arrangement). Non-rigid ear pieces 18 may be disposed opposite one another on inner surface 16 of non-rigid outer shell 12. As shown in the side view of FIG. 4, only one non-rigid ear piece 18 is visible, while the second non-rigid ear piece 18 is concealed on the far side of CPSIA-compliant, sound-deadening infant sleep hat 10.

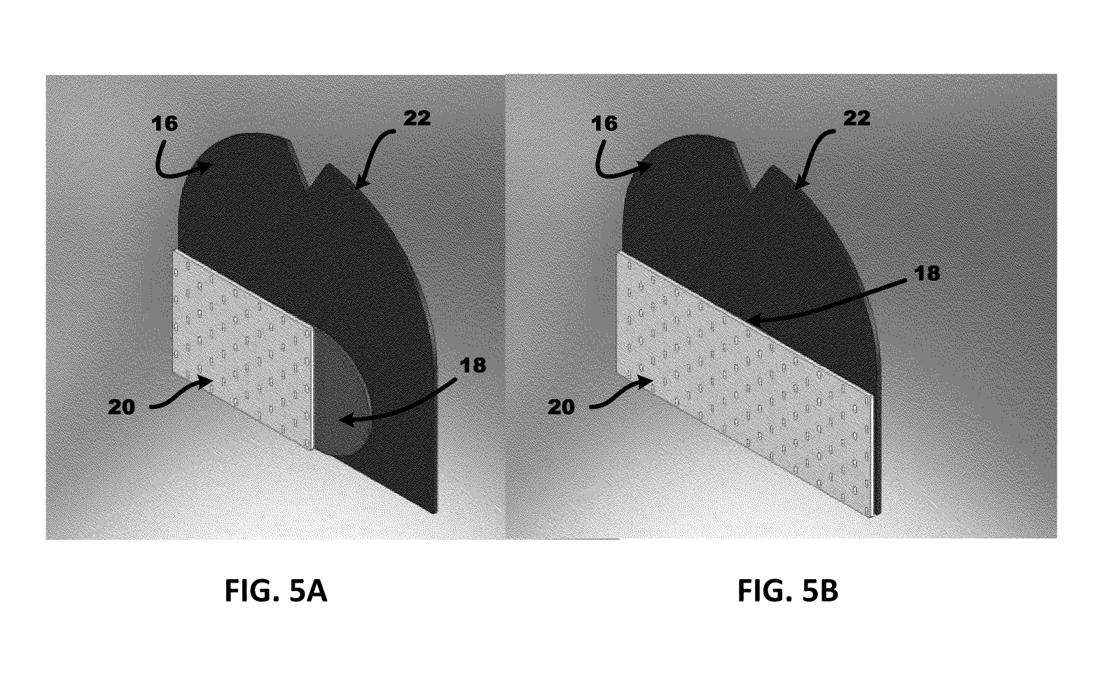

A non-rigid inner band 20 may be disposed over the non-rigid ear pieces on the inner surface of the non-rigid outer shell. As shown in FIG. 4 (and FIGS. 5A and 5B as discussed below), non-rigid inner band 20 may be composed of or include a mesh material (which may comprise polyester or any other suitable material) or may otherwise include voids or perforations that permit heat from an infant's head to pass through non-rigid inner band 20 and contact non-rigid ear pieces 18. In such embodiments, the mesh or otherwise perforated nature of non-rigid inner band 20 abutting non-rigid ear pieces 18 may permit heat from the infant's head to warm the one or more sound-deadening materials disposed behind non-rigid inner band 20 in the non-rigid ear pieces 18. The mesh or perforated nature of non-rigid inner band 20 facilitates greater contact between the infant's head and the sound-deadening material, which in turns facilitates the passage of heat. Upon warming, one or more of the sound-deadening materials (e.g., open-celled foam) softens and conforms inwardly into the infant's ear to block the ear canal from external sound. Where non-rigid ear pieces 18 each include a plurality of layered sound-deadening materials, the sound-deadening material layer of non-rigid ear piece 18 that directly abuts non-rigid inner band 20 may soften and conform inwardly into the infant's ear. In some instances, a portion or all of the layered sound-deadening materials may soften and confirm inwardly into the infant's ear.

FIG. 5A is a perspective view of an exemplary CPSIA-compliant, sound-deadening infant sleep hat showing inner surface 16 of a panel 22 of non-rigid outer shell 12. As shown in FIG. 5A, non-rigid outer shell 12 may include one or more panels 22. FIG. 5A further shows non-rigid inner band 20, which in the shown embodiment comprises half of the total non-rigid inner band 20, and one of the non-rigid ear pieces 18 sandwiched between the non-rigid inner band 20 and panel 22 of non-rigid outer shell 12. As shown in FIG. 5A, part of non-rigid inner band 20 is transparent for the purposes of illustrating the location of non-rigid ear piece 18. FIG. 5B is a perspective view of the exemplary CPSIA-compliant, sound-deadening infant sleep hat of FIG. 1 showing the same components as FIG. 5A with the non-rigid inner band 20 fully displayed. Although FIGS. 5A and 5B depict non-rigid ear piece 18 as a single layer for purposes of illustration, non-rigid ear piece 18 may include one or more sound-deadening materials in a combined or layered arrangement, such as a viscoelastic energy-absorbing foam, a viscoelastic energy-absorbing foam combined with or layered in combination with a one or more fibers (e.g., a polyester fiber blend, reinforced polyester fibers, and the like) material, a viscoelastic energy-absorbing foam combined with or layered in combination with a fabric material, or a viscoelastic energy-absorbing foam combined with or layered in combination with other non-rigid materials that exhibit sound-deadening characteristics.

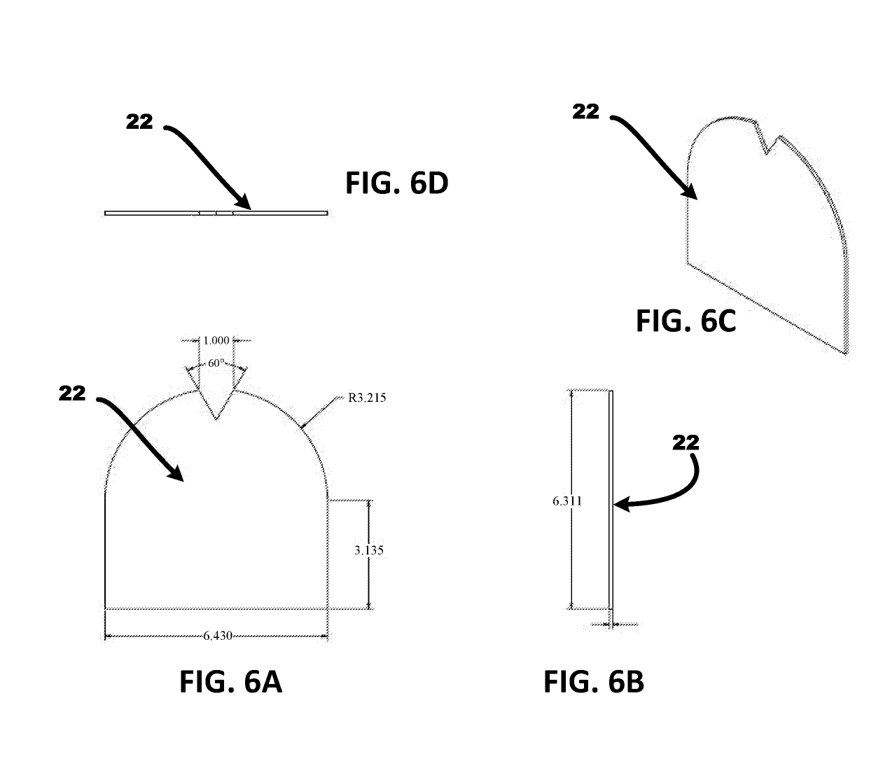

FIGS. 6A through 6D are, respectively, front, side, perspective, and top views of an exemplary panel 22 of non-rigid outer shell 12. Two identical panels 22 may be stitched or otherwise coupled together to form non-rigid outer shell 12. The number of panels 22 used may vary (e.g., one panel 22, two panels 22, three panels 22, and so on) depending on various design specifications (e.g., desired final dimensions), manufacturing considerations (e.g., available manufacturing equipment), the non-rigid materials used for non-rigid outer shell 12, and other variables.

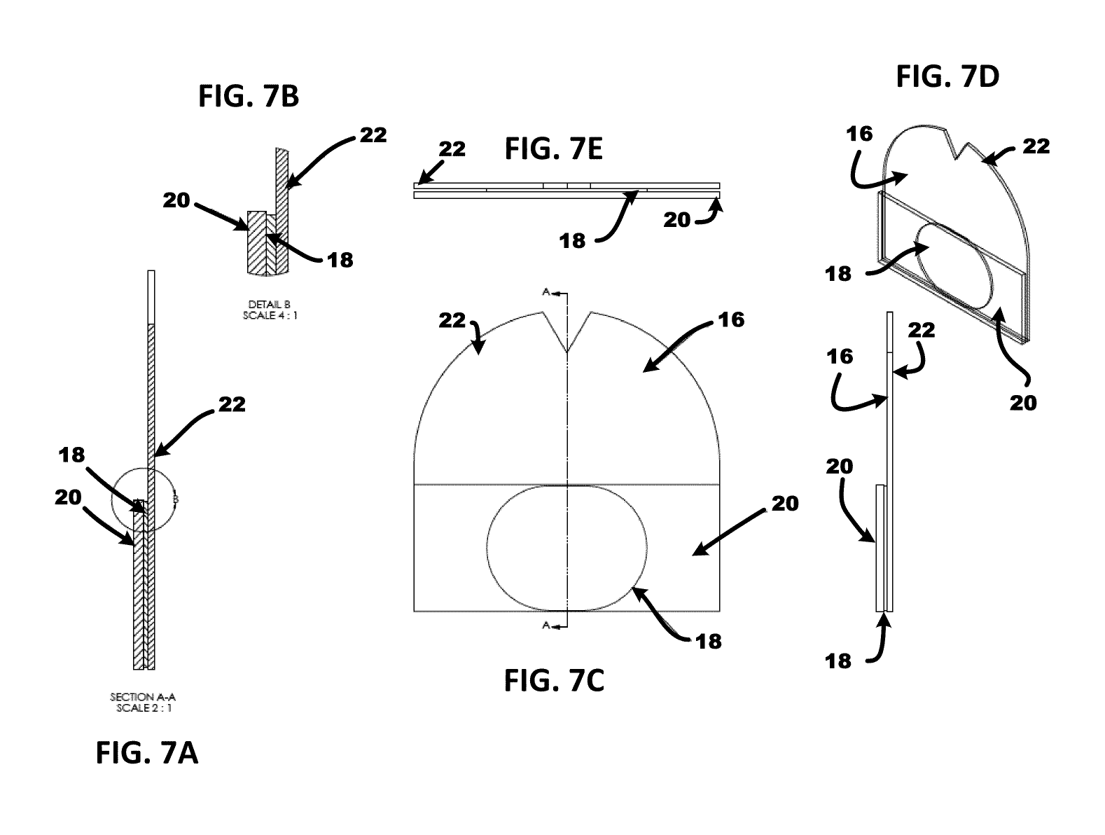

FIGS. 7A through 7E are, respectively, side, zoomed side, front, perspective, and top views of an exemplary panel 22 of non-rigid outer shell 12 combined with an exemplary non-rigid inner band 20, which in the shown embodiment comprises half of the total non-rigid inner band 20, and one of the non-rigid ear pieces 18 sandwiched between the non-rigid inner band 20 and panel 22 of non-rigid outer shell 12. Although FIGS. 7A through 7E depict non-rigid ear piece 18 as a single layer for purposes of illustration, non-rigid ear piece 18 may include one or more sound-deadening materials in a combined or layered arrangement, such as a viscoelastic energy-absorbing foam, a viscoelastic energy-absorbing foam combined with or layered in combination with a one or more fibers (e.g., a polyester fiber blend, reinforced polyester fibers, and the like) material, a viscoelastic energy-absorbing foam combined with or layered in combination with a fabric material, or a viscoelastic energy-absorbing foam combined with or layered in combination with other non-rigid materials that exhibit sound-deadening characteristics.

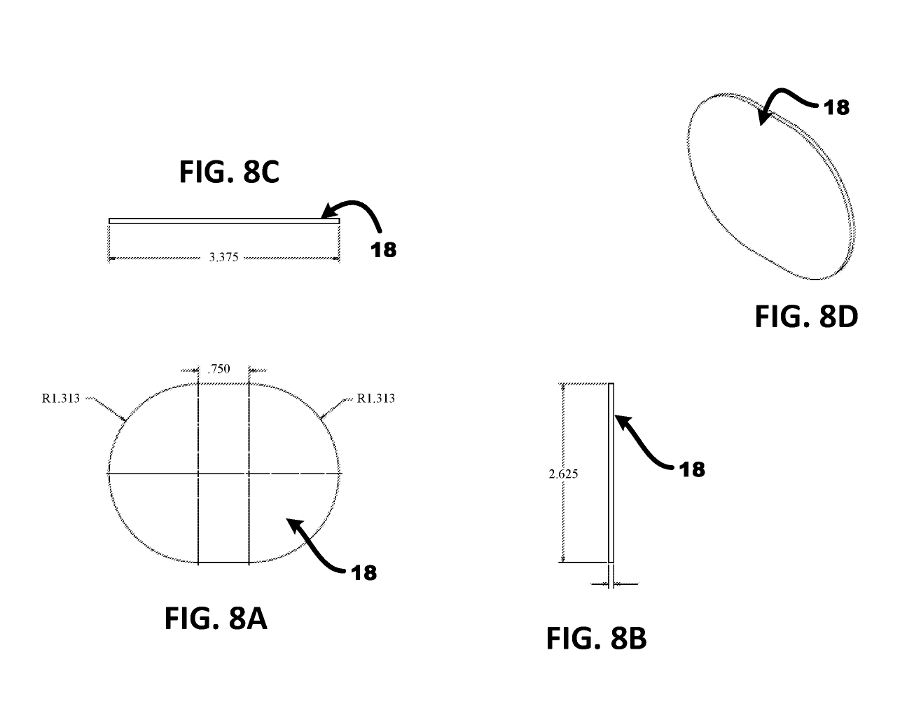

FIGS. 8A through 8D are, respectively, front, side, bottom, and perspective views of an exemplary non-rigid ear piece 18. As discussed above, each non-rigid ear piece 18 may include or be manufactured entirely of one or more sound-deadening materials. The sound-deadening material may be non-rigid to maintain the overall non-rigid construction of non-rigid ear piece 18, thus ensuring that CPSIA-compliant, sound-deadening infant sleep hat 10 is suitable for infant sleep activities. Non-rigid ear pieces 18 may also be soft and non-abrasive against human skin, thereby further increasing the suitability of CPSIA-compliant, sound-deadening infant sleep hat 10 for infant sleep activities. As shown in FIGS. 8A through 8D, non-rigid ear piece 18 may be comprised of a single piece of material, which in some embodiments may be flat and may exclude any recesses or voids so as to increases the ability of the sound-deadening material to conform inwardly against non-rigid inner band 20 and into the ear canal of the infant (thus enhancing the sound-deadening properties of infant sleep hat 10). In other embodiments, non-rigid ear piece 18 may include a plurality of materials, including a plurality of sound-deadening materials in a combined or layered arrangement that similarly conform inwardly into the ear canal of the infant.

Non-rigid ear pieces 18 may vary in size, thickness, and shape depending on the overall size and design of CPSIA-compliant, sound-deadening infant sleep hat 10. In some embodiments, non-rigid ear pieces 18 may each include a solid area of foam or other soft material that deadens sound without requiring a seal around the infant's ear. In one embodiment, for instance, the one or more sound-deadening material of non-rigid ear pieces 18 may include a viscoelastic energy-absorbing foam, such as Confor® foam manufactured by Aearo Technologies LLC of Indianapolis, Ind. The sound-deadening material may be an open-celled polyurethane foam. In some embodiments, the sound-deadening material may be a non-memory foam. By not creating a complete seal around an infant's ear, non-rigid ear pieces 18 avoid causing excess pressure that can cause irritation and discomfort over time. The choice of foam or other sound-deadening materials may vary depending on cost considerations, availability, and the overall size and design of CPSIA-compliant, sound-deadening infant sleep hat 10. As noted above, the sound-deadening material may be non-rigid, capable of reducing or deadening noise, non-irritating and comfortable against human skin, and compliant with U.S. Consumer Product Safety Commission child sleepwear regulations promulgated pursuant to the CPSIA.

In various embodiments, CPSIA-compliant, sound-deadening infant sleep hat 10 may be constrained to a size sufficient to snugly fit over an infant's head without applying harmfully high pressure to the infant's ears. For example, CPSIA-compliant, sound-deadening infant sleep hat 10 may have a circumference ranging from 37 to 43 cm and a height ranging from 11.5 to 17 cm. The proper sizing of sound-deadening infant sleep hat 10 permits it to remain in place upon an infant head without the need for tassels, straps, headband-like components, or other potential choking hazards.

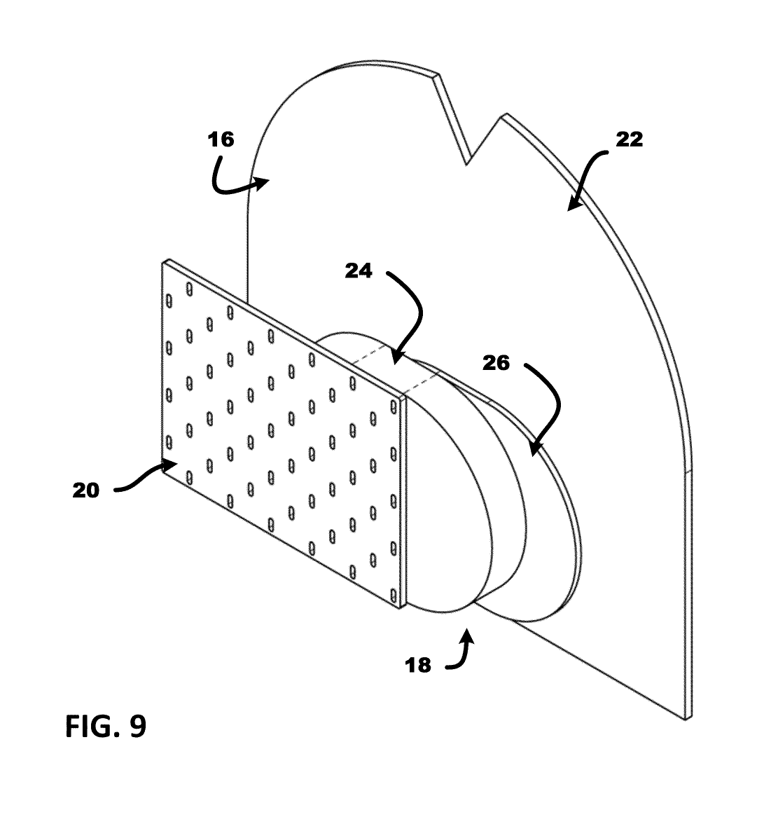

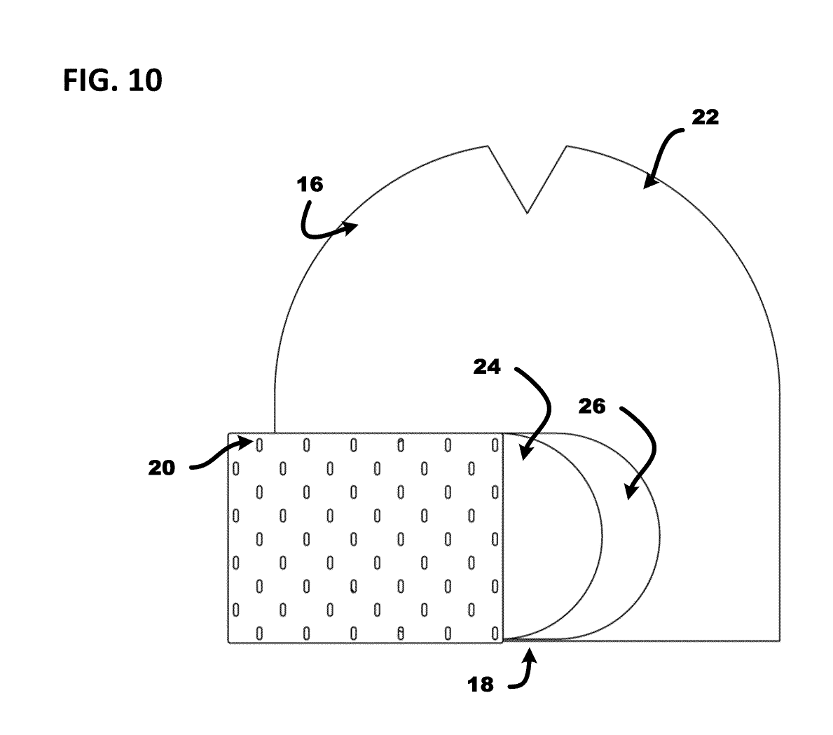

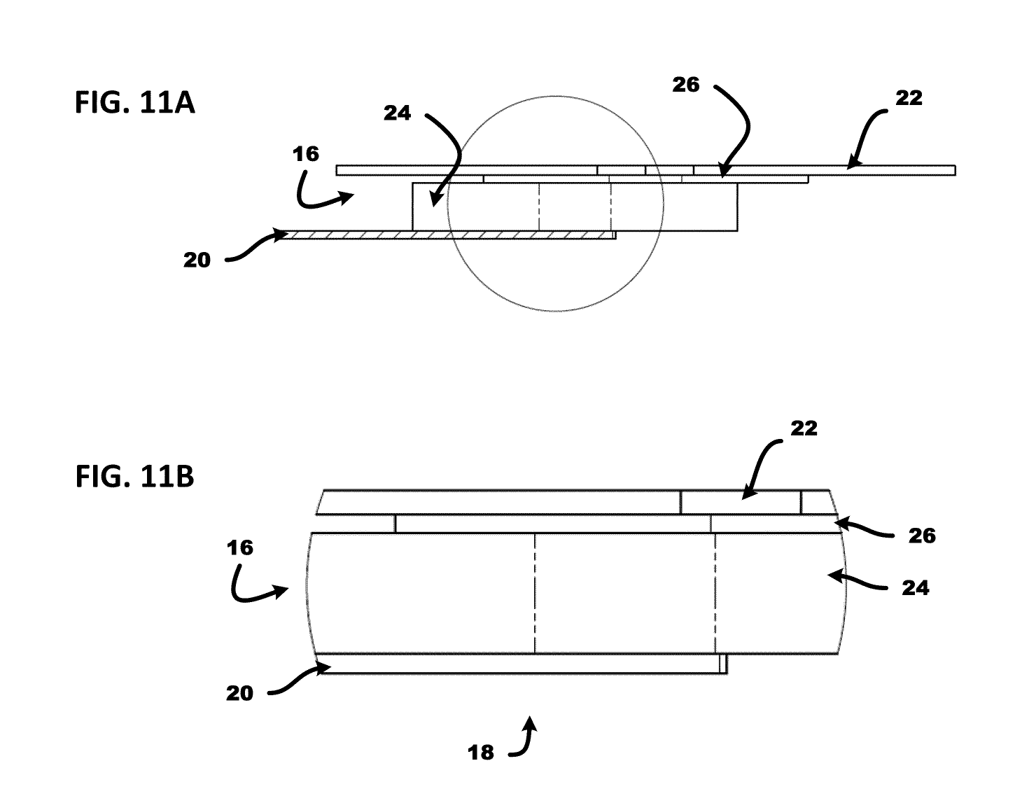

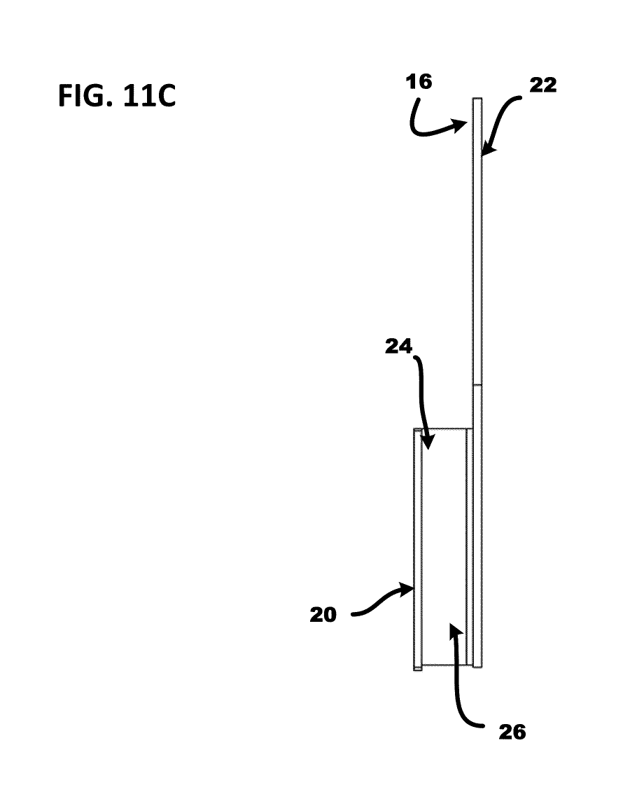

FIGS. 9, 10, 11A, 11B, and 11C are, respectively, perspective, front, top, zoomed top, and side views of an exemplary panel 22 of a non-rigid outer shell 12 combined with an exemplary non-rigid inner band 20 and an exemplary non-rigid ear piece 18. Non-rigid ear piece 18 disposed under non-rigid inner band 20 (e.g., mesh band) may, in some instances, include a plurality of sound-deadening materials in a combined or layered arrangement, such as a viscoelastic energy-absorbing foam, a viscoelastic energy-absorbing foam combined with or layered in combination with a material composed at least partially of one or more fibers (e.g., a polyester fibers, a reinforced fiber blend, a reinforced polyester fiber blend, and the like), a viscoelastic energy-absorbing foam combined with or layered in combination with a fabric material, or a viscoelastic energy-absorbing foam combined with or layered in combination with other non-rigid materials that exhibit sound-deadening characteristics. The foam or other sound-deadening materials absorb and disperse sound waves to varying degrees depending on the properties of the selected foam or other sound-deadening material.

As shown in the exemplary embodiment of FIG. 9, the CPSIA-compliant, sound-deadening infant sleep hat may include a multipart non-rigid ear piece 18. Non-rigid ear piece 18 may include at least a first non-rigid layer 24 and a second non-rigid layer 26. Both first non-rigid layer 24 and second non-rigid layer 26 may include one or more sound-deadening materials. In one embodiment, first non-rigid layer 24 may be composed of one or more fibers (e.g., a polyester fiber blend, reinforced polyester fibers, and the like), while second non-rigid layer 26 may be composed of a viscoelastic energy-absorbing foam (e.g., open-celled viscoelastic foam). In such an embodiment, first non-rigid layer 24 may serve as a fiber backing to second non-rigid layer 26. In such an embodiment, the combination of first non-rigid layer 24 composed of one or more fibers with second non-rigid layer 26 composed of a viscoelastic energy-absorbing foam provides CPSIA-compliant, sound-deadening infant sleep hat with enhanced sound-deadening capabilities.

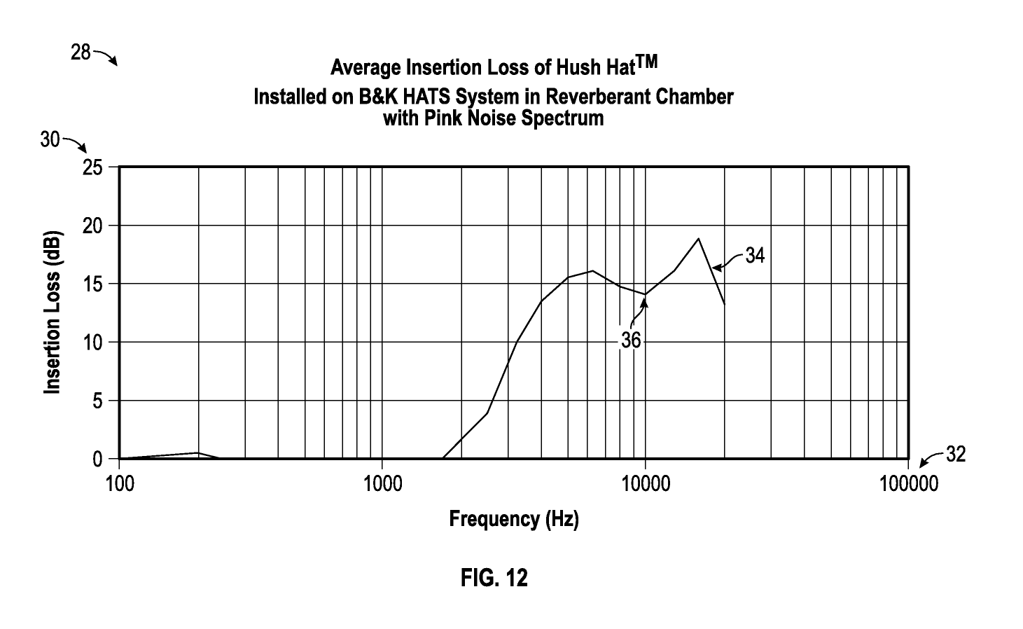

FIG. 12 is a graph that illustrates the sound-deadening characteristics of an exemplary CPSIA-compliant, sound-deadening infant sleep hat. Graph 28 reports the results of sound-deadening testing performed on an exemplary CPSIA-compliant, sound-deadening infant sleep hat in which non-rigid ear pieces 18 are composed of a viscoelastic sound-deadening foam (e.g., as depicted in FIGS. 5A and 5B). Graph 28 reports the sound-deadening data as average sound insertion loss (dB) along Y-axis 30 versus audio signal frequency (Hz) along X-axis 32. The sound-deadening data was collected using an exemplary CPSIA-compliant, sound-deadening infant sleep hat installed and tested on a Brüel & Kjar Head and Torso Simulator (B&K HATS) system in a reverberant chamber using pink noise.

As illustrated by graph 28, the exemplary CPSIA-compliant, sound-deadening infant sleep hat tested is capable of producing a sound insertion loss ranging from at least around 1 dB to around 20 dB over a frequency range from at least around 1.5 kHz to around 20 kHz. The exemplary CPSIA-compliant, sound-deadening infant sleep hat tested is capable of producing a sound insertion loss of at least 5 dB over a frequency range from at least around 11.5 kHz to around 20 kHz. The exemplary CPSIA-compliant, sound-deadening infant sleep hat tested is capable of producing a sound insertion loss of at least 10 dB over a frequency range from at least around 12.3 kHz to around 20 kHz. Graph 28 further illustrates that the exemplary CPSIA-compliant, sound-deadening infant sleep hat tested is capable of producing a sound insertion loss of at least around 1 dB over a frequency range from at least around 150 kHz to around 250 kHz. As illustrated by peak 34 indicated on graph 28, the exemplary CPSIA-compliant, sound-deadening infant sleep hat tested is capable of effecting a sound insertion loss of at least around 18 dB in audio signal having a frequency of around 15 kHz. As indicated at data point 36 of graph 28, the exemplary CPSIA-compliant, sound-deadening infant sleep hat tested is capable of producing a sound insertion loss of at least around 14 dB in an audio signal having a frequency of around 10 kHz.

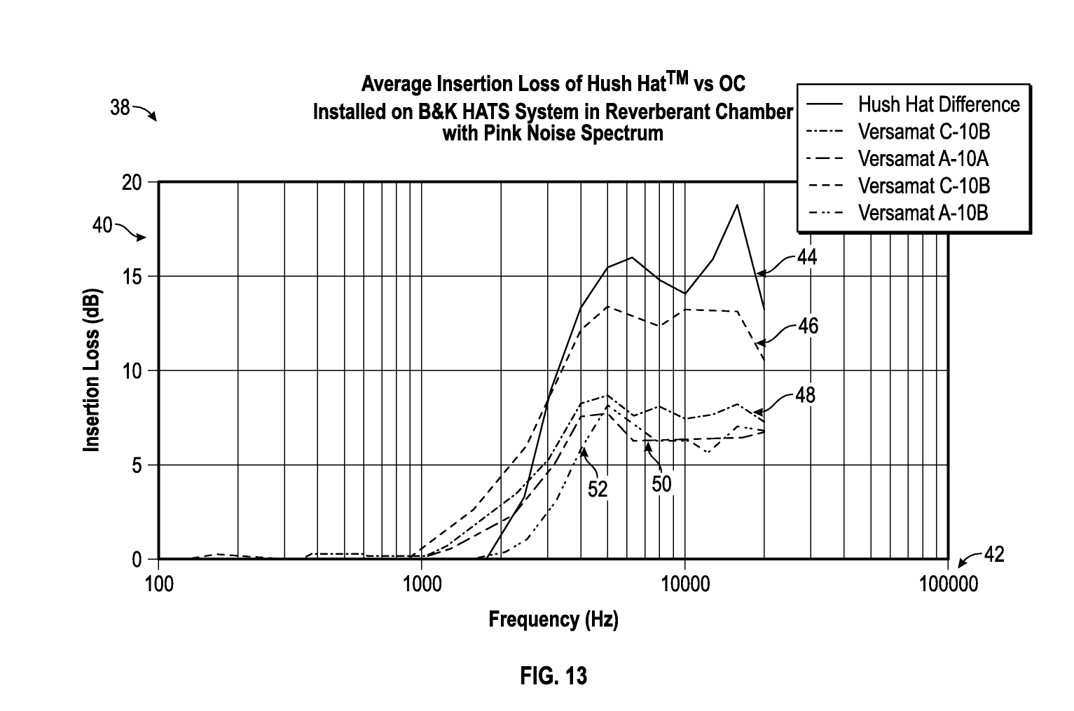

FIG. 13 is another graph that illustrates the sound-deadening characteristics of an exemplary CPSIA-compliant, sound-deadening infant sleep hat. Graph 38 reports the result of sound-deadening testing performed on an exemplary CPSIA-compliant, sound-deadening infant sleep hat in which non-rigid ear pieces 18 are composed of a viscoelastic sound-deadening foam (e.g., as depicted in FIGS. 5A and 5B). Graph 38 reports the sound-deadening data as average insertion loss (dB) along Y-axis 40 versus audio signal frequency (Hz) along X-axis 42. Graph 38 depicts sound-deadening data for the CPSIA-compliant, sound-deadening infant sleep hat (represented by line 44) along with sound-deadening data for a plurality of sound-deadening materials that are at least partially composed of one or more fibers (e.g., a polyester fiber blend, reinforced polyester fibers, and the like). A first exemplary sound-deadening material at least partially composed of at least one or more fibers represented by line 46, is a first VersaMat® material having a first density and thickness produced by Owens Corning. A second sound-deadening material at least partially composed of one or more fibers (e.g., a polyester fiber blend, reinforced polyester fibers, and the like), represented by line 48, is a second Versamat® material having a second density and thickness. A third sound-deadening material at least partially composed of one or more fibers (e.g., a polyester fiber blend, reinforced polyester fibers, and the like) represented by line 50, is a third Versamat® material having a third density and thickness. A fourth sound-deadening material at least partially composed of one or more fibers (e.g., a polyester fiber blend, reinforced polyester fibers, and the like) represented by line 52, is a fourth Versamat® material having a fourth density and thickness. As illustrated by line 44 of graph 38, the exemplary CPSIA-compliant, sound-deadening infant sleep hat may effectively deaden sounds in the around 1.5 kHz and above range (i.e., “high pitch” sounds like a phone ringing, a vacuum running, or birds chirping) by way of non-rigid ear pieces 18 including a viscoelastic energy-absorbing foam (e.g., Confor® foam).

As illustrated by lines 46, 48, 50, and 52 of graph 38, the exemplary CPSIA-compliant, sound-deadening infant sleep hat may effectively deaden sounds at lower frequencies (e.g., “low pitch” sounds like a typical human voice, a lawn mower or car engine, or a dog barking) by way of including a sound-deadening material at least partially composed of one or more fibers (e.g., a polyester fiber blend, reinforced polyester fibers, and the like), such as a Versamat® material). As shown at line 46, for example, the first sound-deadening material at least partially composed of one or more fibers was capable of effecting an insertion loss ranging between at least around 1 dB to around 7.5 dB over a frequency range of at least around 850 Hz to around 1.2 kHz. Thus, by combining the viscoelastic energy-absorbing foam with one or more materials at least partially composed of one or more fibers (e.g., a polyester fiber blend, reinforced polyester fibers, and the like, such as Versamat® materials of varying densities and thicknesses as illustrated in graph 38), the CPSIA-compliant, sound-deadening infant sleep hat disclosed herein may be customized to deaden sound over a wide range of frequencies (e.g., between around 1 dB to around 20 dB over a frequency of at least around 850 Hz to at least around 20 kHz) or to intelligently deaden sound only at desired frequencies.

An exemplary manufacturing method for creating CPSIA-compliant, sound-deadening infant sleep hat 10 of FIG. 1 may include folding the right sides of a plurality of panels together in a lengthwise orientation (such as those shown in FIGS. 5A and 5B). The method may include sewing or otherwise coupling darts on each panel. The right sides of both panels may be sewn together around an outer perimeter while leaving a small opening (e.g., 1″-1.25″). The method may include turning the coupled panels, which may form non-rigid outer shell 12 of FIG. 1, right side out through the small opening and folding the panels to place the inner half of non-rigid outer shell inside the outer hat. The method may also include matching darts and seam lines and making sure that the opening is disposed in the interior of non-rigid outer shell 12 of FIG. 1. The method may further include stitching the small seam closed with a seam allowance tucked under.

A top stitch may be applied ¼″ around darts and seams at the top of the hat. The method may also include stitching 2″ down each seam side. The method may include matching the long sides together and creating a stitch while leaving a 2″ opening. The assembled hat right may be turned side out. The method may include inserting a non-rigid ear piece into a liner disposed ½″ back from a center line. The raw edges of the short sides may be brought through the 2″ opening created earlier in the process. The method may include matching and then stitching the edges and seams. The new seam may be pulled back through the opening. In some embodiments, a circle with a small opening along the lower seam line may be visible.

The method may include inserting a second non-rigid ear piece through the opening and positioning the ear piece at the vertical seam. The method may include folding the in seam allowance and stitching the opening closed. The method may further include compressing the non-rigid ear piece and pinning it away from the seam allowance. With the right sides together, the top and bottom pieces may be pinned and stitched together. In some embodiments, the method may include ensuring that the non-rigid ear piece is not caught in the stitching. The method may include folding the lining into the center of the assembled hat. The method may also include stitching a label, which may be applied either to the inner or outer surface of non-rigid outer shell. The method may include stitching through all layers of the assembled hat on the left and right sides of the label to securely fix it in place. Persons of ordinary skill in the art will readily appreciate that the foregoing method is exemplary and that other methods of creating the CPSIA-compliant, sound-deadening infant sleep hat disclosed herein are possible and within the scope of the present disclosure. The foregoing steps may be performed in a variety of possible chronological orders and with a variety of possible materials and dimensions.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A sound-deadening infant sleep hat, the hat comprising:
a non-rigid outer shell having an inner surface; and
two non-rigid ear pieces disposed opposite one another on the inner surface of the non-rigid outer shell, the non-rigid ear pieces each composed at least partially of at least one sound-deadening material.

2. The sound-deadening infant sleep hat of claim 1, wherein the at least one sound-deadening material is a viscoelastic foam.

3. The sound-deadening infant sleep hat of claim 2, wherein the viscoelastic foam is open-celled.

4. The sound-deadening infant sleep hat of claim 1, further comprising a non-rigid inner band disposed over the two non-rigid ear pieces on the inner surface of the non-rigid outer shell.

5. The sound-deadening infant sleep hat of claim 4, wherein the non-rigid inner band includes a plurality of voids.

6. The sound-deadening infant sleep hat of claim 4, wherein the non-rigid band is composed at least partially of mesh.

7. The sound-deadening infant sleep hat of claim 1, wherein each non-rigid ear piece includes a first sound-deadening material and a different second sound-deadening material.

8. The sound-deadening infant sleep hat of claim 7, wherein the first sound-deadening material is a viscoelastic foam and the second sound-deadening material is composed at least partially of one or more fibers.

9. The sound-deadening infant sleep hat of claim 8, further comprising a mesh band disposed over the two non-rigid ear pieces on the inner surface of the non-rigid outer shell.

10. The sound-deadening infant sleep hat of claim 1, wherein each non-rigid ear piece is capable of effecting a sound insertion loss of at least 18 dB in an audio signal having a frequency of 15 kHz.

11. The sound-deadening infant sleep hat of claim 9, wherein each non-rigid ear piece is capable of effecting a sound insertion loss of at least 18 dB in an audio signal having a frequency of 15 kHz.

12. A sound-deadening infant sleep hat, the hat comprising:
a non-rigid outer shell having an inner surface; and
two non-rigid ear pieces disposed opposite one another on the inner surface of the non-rigid outer shell, the non-rigid ear pieces each at least partially composed of at least a first sound-deadening material and a different second sound-deadening material.

13. The sound-deadening infant sleep hat of claim 12, wherein the first sound-deadening material is a viscoelastic foam and the second sound-deadening material is composed at least partially of one or more fibers.

14. The sound-deadening infant sleep hat of claim 13, wherein the viscoelastic foam is open-celled.

15. The sound-deadening infant sleep hat of claim 13, further comprising a non-rigid inner band disposed over the two non-rigid ear pieces on the inner surface of the non-rigid outer shell.

16. The sound-deadening infant sleep hat of claim 15, wherein the non-rigid inner band includes a plurality of voids.

17. The sound-deadening infant sleep hat of claim 15, wherein the non-rigid band is composed at least partially of mesh.

18. The sound-deadening infant sleep hat of claim 12, wherein each non-rigid ear piece is capable of effecting a sound insertion loss of at least 18 dB in an audio signal having a frequency at 15 kHz and a sound insertion loss of at least 1 dB at a frequency of 1 kHz.

19. The sound-deadening infant sleep hat of claim 13, wherein each non-rigid ear piece is capable of effecting a sound insertion loss of at least 18 dB in an audio signal having a frequency of 15 kHz and a sound insertion loss of at least 1 dB at a frequency of 1 kHz.

20. A sound-deadening infant sleep hat, the hat comprising:
a non-rigid outer shell having an inner surface;
two non-rigid ear pieces disposed opposite one another on the inner surface of the non-rigid outer shell, the non-rigid ear pieces each at least partially composed of at least a first layer of viscoelastic sound-deadening foam and a second layer of sound-deadening material composed at least partially of one or more fibers; and
a mesh band disposed over the two non-rigid ear pieces on the inner surface of the non-rigid outer shell.
